(19) United States
(12) Patent Application Publication
(10) Pub. No.: US 2016/0113808 A1
(43) Pub. Date: Apr. 28, 2016

(54) STERNUM SUPPORTING HARNESS

(71) Applicant: ZENTAN TECHNOLOGY CO., LTD., NEW TAIPEI CITY (TW)

(72) Inventor: TONG-PIE CHEN, NEW TAIPEI CITY (TW)

(21) Appl. No.: 14/657,599

(22) Filed: Mar. 13, 2015

(30) Foreign Application Priority Data
Oct. 22, 2014 (TW) 103218765

(51) Int. Cl. A61F 5/03

(52) U.S. Cl. CPC A61F 5/03

(57) ABSTRACT

A sternum supporting harness includes a carrying strap structure and a chest strap structure surroundingly disposed thereon. The carrying strap structure includes a main body, a shoulder portion, including a left shoulder strap and a right shoulder strap, a flank portion, and an adjustment portion. One end of the left shoulder strap and one end of the right shoulder strap are connected to two upper end portions of the main body respectively. Another end of the left shoulder strap and another end of the right shoulder strap are both connected to the flank portion. The adjustment portion is connected to the flank portion. The chest strap structure includes a grabbing unit and a belt-like unit connected thereto. The grabbing unit includes a first grabbing portion, a second grabbing portion, and a base portion.

### BACKGROUND OF THE INVENTION

1. Field of the Invention

The present disclosure relates to a supporting harness; in particular, to a sternum supporting harness for supporting a sternum.

2. Description of Related Art

Generally, a sternum supporting harness is configured for facilitating a patient after cardiac surgery. The patient's sternum after surgery has not been repaired completely and still need a few months of recovery; in this period of time, the patient needs to wear a sternum supporting harness to prevent the sternum from displacement.

Traditional sternum supporting harnesses are formed in one-piece and have two grabbing rings, and the sternum supporting harness can support the sternum when the patient grabs the two grabbing rings. One-piece formed sternum supporting harnesses need to be worn outside the clothing of the patient, which results in a strange look from strangers because of the unexpected appearance of the sternum supporting harness on the patient. This gives the patient stress psychologically.

Traditional sternum supporting harnesses are configured only for supporting the user's sternum. However, the condition of the patient after cardiac surgery needs to be further observed and the patient's physiological information needs to be tracked. The patient needs to wear an additional signal transmission belt to detect physiological information.

Therefore, how to provide a sternum supporting harness that can be worn inside the user's clothing, so the user does not receive any strange looks, and one that also is able to detect the user's physiological information (such as heart rate) so the recovery condition of the user can be observed and if the user's cardiac rhythm is under normal condition can be checked through the physiological information has become an important issue.

### SUMMARY OF THE INVENTION

The present disclosure provides a sternum supporting harness which is configured to be worn inside the user's clothing, which allows the user not to be subject to looks from strangers. In addition, the sternum supporting harness is configured to detect the user's physiological information such as information of the user's pulsating heartbeat, allowing for the observation of the user's physiological condition and for the determination of the normality of the user's cardiac rhythm.

The present disclosure provides a sternum supporting harness for supporting a sternum. The sternum supporting harness includes a carrying strap structure and a chest strap structure detachably attached on the carrying strap structure. The carrying strap structure includes a main body, a shoulder portion, a flank portion, and an adjustment portion. The main portion has two upper end portions respectively positioned at opposite sides of the main body. The shoulder portion includes a left shoulder strap and a right shoulder strap. An end of the left shoulder strap and an end of the right shoulder strap are connected to the two upper end portions respectively. Another end of the left shoulder strap and another end of the right shoulder strap are both connected to the flank portion. The adjustment portion is connected to the flank portion for facilitating the main body to be disposed on the sternum.

The chest strap structure includes a grabbing unit and a belt-like unit. The grabbing unit is connected to the belt-like unit. The chest strap structure is surroundingly disposed on the carrying strap structure through the belt-like unit and the grabbing unit. The grabbing unit includes a first grabbing portion, a second grabbing portion, and a base portion. The first grabbing portion and the second grabbing portion are correspondingly arranged with respect to each other, and the first grabbing portion and the second grabbing portion are respectively positioned at two opposite sides of the base portion. The sternum supporting harness is for supporting the sternum when the first grabbing portion and the second grabbing portion are grabbed.

The sternum supporting harness according to the present disclosure including the carrying strap structure and the chest strap structure detachably attached thereto can be two-piece worn on the user. The sternum supporting harness can support the user's sternum to prevent the wound from being torn and to prevent the chest from fracturing when the user grabs the first grabbing portion and the second grabbing portion. The sternum supporting harness is configured for detecting and recording the user's physiological information. Moreover, the sternum supporting harness can be used in conjunction with an electrode structure disposed on the flank portion for monitoring the user's physiological information after surgery. Furthermore, the sternum supporting harness, which includes the carrying strap structure and the chest strap structure, is configured to be two-piece worn on the user and utilizes the first grabbing portion and the second grabbing portion to simplify the design, therefore the sternum supporting harness can be worn inside the user's clothing and in direct contact with the user's skin, for providing preferred support to the user's sternum, and the user does not receive any strange looks.

In order to further the understanding regarding the present disclosure, the following embodiments are provided along with illustrations to facilitate the disclosure of the present disclosure.

### BRIEF DESCRIPTION OF THE DRAWINGS

FIG. 1A shows a perspective view of a carrying strap structure according to an embodiment of the present disclosure;

FIG. 1B shows another perspective view of the carrying strap structure according to an embodiment of the present disclosure;

FIG. 1C shows a front view of the carrying strap structure according to an embodiment of the present disclosure;

FIG. 1D shows a rear view of the carrying strap structure according to an embodiment of the present disclosure;

FIG. 1E shows an exploded view of a combination of a signal transceiver and a clasp assembly;

FIG. 2A shows a perspective view of a chest strap structure according to an embodiment of the present disclosure;

FIG. 2B shows a front view of the chest strap structure according to an embodiment of the present disclosure;

FIG. 3A shows a perspective view of a sternum supporting harness according to an embodiment of the present disclosure;

FIG. 3B shows a perspective view of the sternum supporting harness in use according to an embodiment of the present disclosure; and

FIG. 3C shows another perspective view of the sternum supporting harness in use according to an embodiment of the present disclosure.

### DETAILED DESCRIPTION OF THE PREFERRED EMBODIMENTS

The aforementioned illustrations and following detailed descriptions are exemplary for the purpose of further explaining the scope of the present disclosure. Other objectives and advantages related to the present disclosure will be illustrated in the subsequent descriptions and appended drawings.

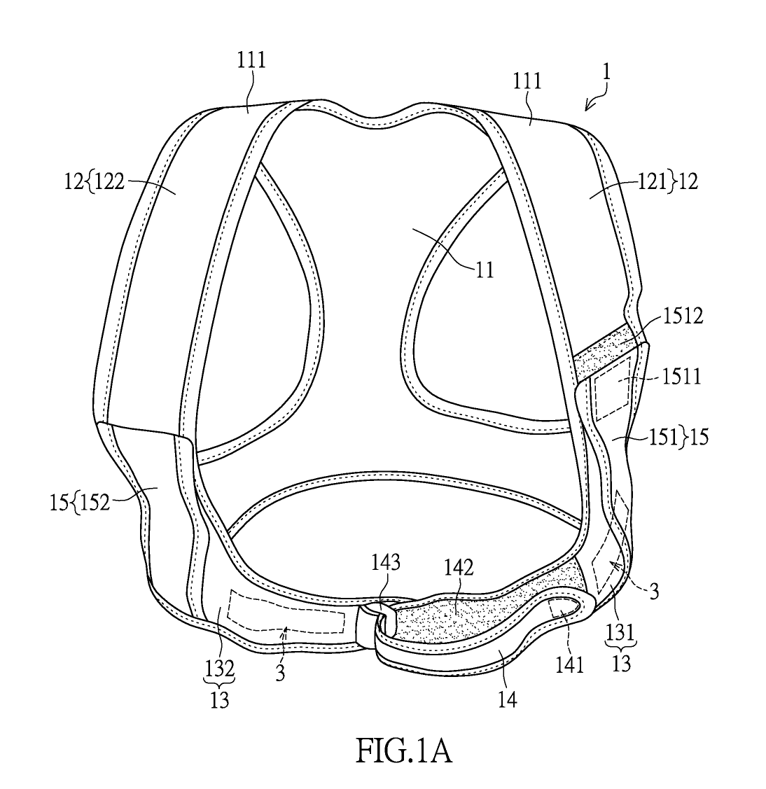

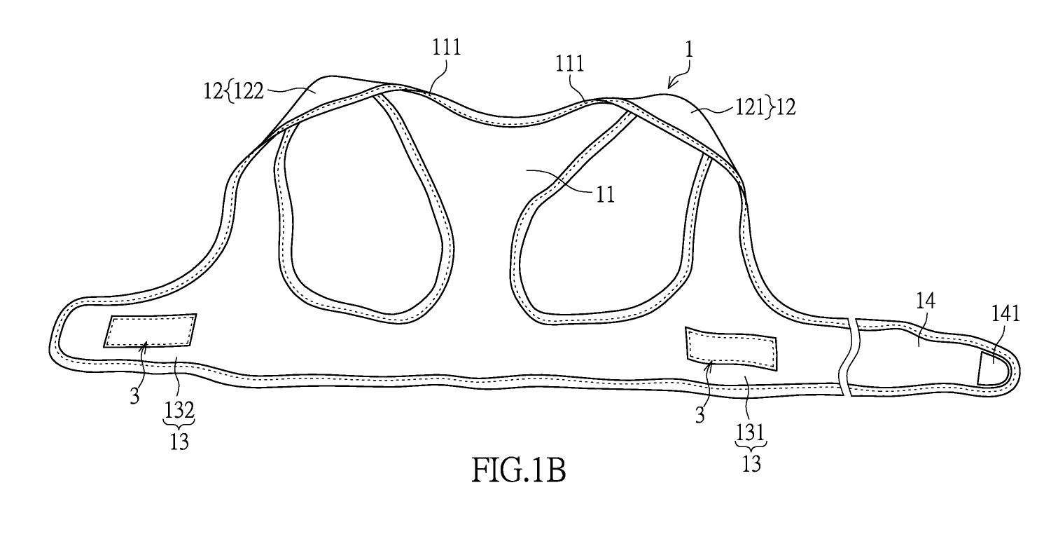

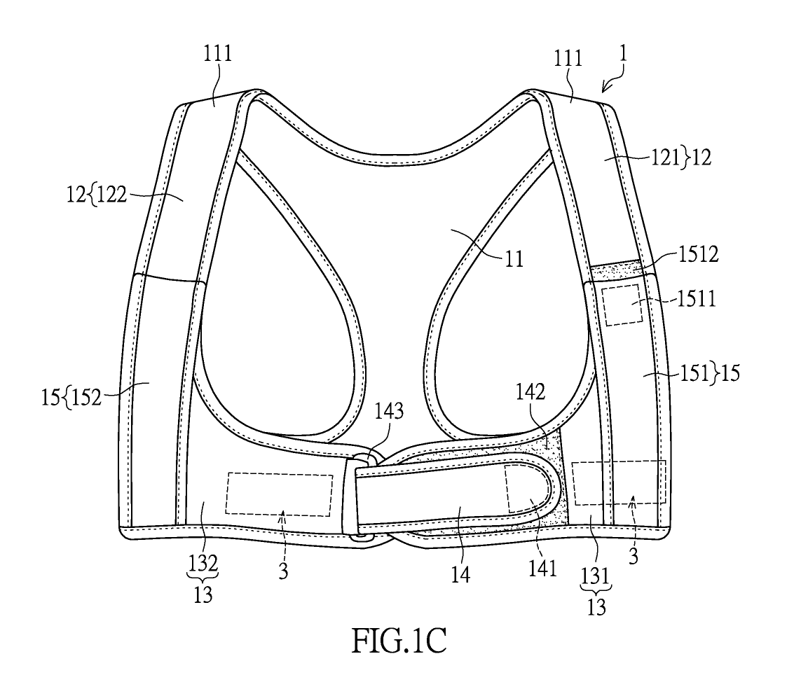

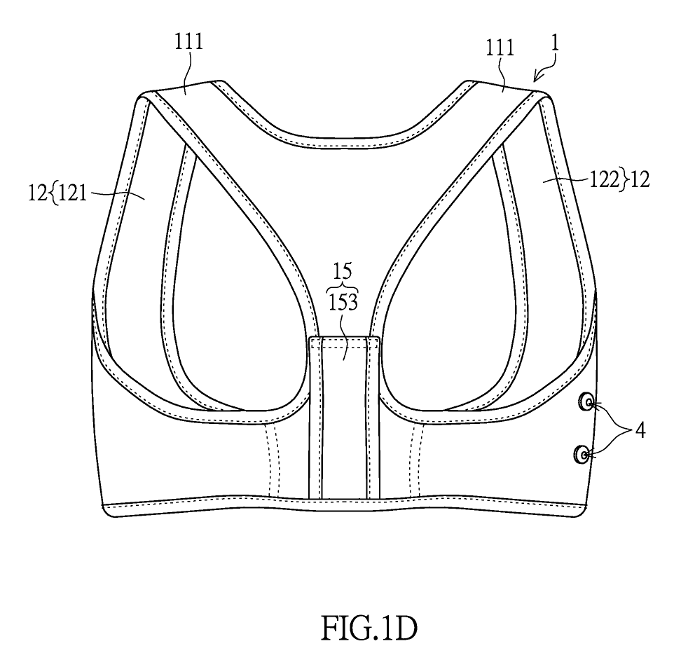

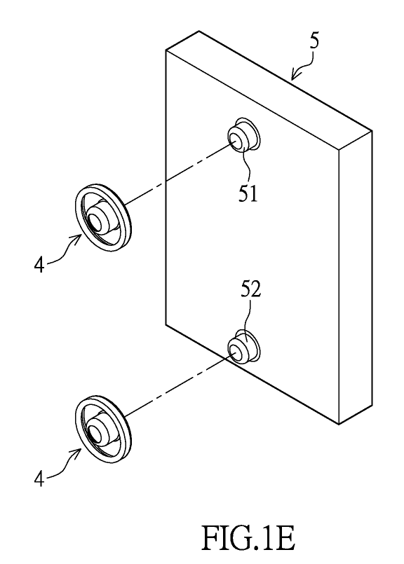

The present disclosure provides a sternum supporting harness S, which is configured to support a sternum of a user and includes a carrying strap structure 1 and a chest strap structure 2. Please refer to FIG. 1A to FIG. 1D for explanation of the carrying strap structure according to the present disclosure. FIG. 1A shows a perspective view of a carrying strap structure according to an embodiment of the present disclosure. FIG. 1B shows another perspective view of the carrying strap structure according to an embodiment of the present disclosure. FIG. 1C shows a front view of the carrying strap structure according to an embodiment of the present disclosure. FIG. 1D shows a rear view of the carrying strap structure according to an embodiment of the present disclosure. As shown in FIG. 1A and FIG. 1B, the carrying strap structure 1 according to the present disclosure includes a main body 11, a shoulder portion 12, a flank portion 13, and an adjustment portion 14. The shoulder portion 12 includes a left shoulder strap 121 and a right shoulder strap 122. The main body 11 has two upper end portions 111 respectively positioned at opposite sides of the main body 11. One end of the left shoulder strap 121 and one end of the right shoulder strap 122 are respectively connected to the two upper end portions 111. Another end of the left shoulder strap 121 and another end of the right shoulder strap 122 both are connected to the flank portion 13. In addition, the adjustment portion 14 is connected to the flank portion 13 for facilitating the main body 11 to be disposed on the sternum. The adjustment portion 14 enables the user to adjust the carrying strap structure 1 to a desired tightness when the carrying strap structure 1 is worn on the body of the user. The carrying strap structure 1 can be formed as a vest to be worn on the body of the user, such as a patient after surgery, and the present disclosure is not limited thereto. For example, the carrying strap structure 1 can be formed of fabrics having resilience to fit the user. In an exemplary embodiment, the main body 11 can be formed as a vest, and the present disclosure is not limited thereto.

According to the present disclosure, the flank portion 13 includes a left flank strap 131 and a right flank strap 132. Said another end of the left shoulder strap 121 is connected to the left flank strap 131. Said another end of the right shoulder strap 122 is connected to the right flank strap 132. In addition, the flank portion 13 has a retaining ring 143 disposed thereon for use in conjunction with the adjustment portion 14. Specifically, in the instant embodiment, the retaining ring 143 can be disposed on the right flank portion 132 and positioned at a free end of the right flank portion 132. The adjustment portion 14 is connected to the left flank portion 131, and the adjustment portion 14 and the left flank portion 131 can be one-piece formed. Moreover, the adjustment portion 14 can be formed with a first connecting element 141 and a second connecting element 142. The first connecting element 141 and the second connecting element 142 are respectively arranged at two opposite ends of the adjustment portion 14. For example, the first connecting element 141 and the second connecting element 142 can be hook-and-loop fasteners such as Velcro, such that the first connecting element 141 and the second connecting element 142 can be detachably attached to each other. As a specific example, the first connecting element 141 can be a fastener having Velcro hooks, and the second connecting element 142 can be a fastener having Velcro loops, but the present embodiment is not limited thereto. In an exemplary embodiment, the adjustment portion 14 can be formed merely with the first connecting element 141 and the second connecting element 142 and facilitate the carrying strap structure 1 to be disposed on the sternum through the first connecting element 141 and the second connecting element 142.

The first connecting element 141 and the second connecting element 142 can be connected to each other in another configuration. The free end of the adjustment portion 14, which is not connected to the left flank strap 131 and at which the first connecting element 141 is positioned, can be inserted through the aperture of the retaining ring 143 disposed on the right flank strap 132 and secured to the second connecting element 142 via the first connecting element 141. Therefore, the adjustment portion 14 facilitates the carrying strap structure 1 to be disposed on the sternum through the retaining ring 143, the first connecting portion 141, and the second connecting portion 142 and enables the user to adjust the main body 11 to a desired tightness. In another embodiment, the adjustment portion 14 can adapt a snap-fit buckle for connecting, and there need be no retaining ring 143 disposed on the right flank strap 132. Specifically, the adjustment portion 14 can be formed with a male element of the buckle (not shown in the Figs), a female element of the buckle (not shown in the Figs), and an adjustment structure (not shown in the Figs). The male element of the buckle and the adjustment structure can be disposed on the left flank strap 131, and the female element of the buckle can be disposed on the right flank strap 132. The adjustment structure enables the user to adjust the adjustment portion 14 to a desired length according to the chest circumference of the user. Conventional adjustment structure well known to those skilled in the art may be employed and the instant disclosure is not limited thereto.

The carrying strap structure 1 further includes an electrode structure 3 disposed on the flank portion 13. Specifically, in the present embodiment, the carrying strap structure 1 includes two electrodes structures 3 respectively disposed on the left flank strap 131 and the right flank strap 132 for detecting and recording the user's physiological information. It is worth noting that, the electrode structure 3 can be formed of a flexible material. For example, the electrode structure 3 can be formed of an electrically conductive fabric such as a non-woven cloth and bonded onto the flank portion 13 by hot pressing, coating, weaving, printing, molding or other methods. Moreover, the electrode structure 3 can be formed with a pressed strip (not shown in the Figs) peripherally arranged for fixing the electrode structure 3 on the flank portion 13.

Referring to FIG. 1C and FIG. 1D, the carrying strap structure 1 can further include a clasp assembly 4 disposed on the main body 11 and electrically connected to the electrode structure 3. The user's physiological information detected by the electrode structure 3 can be transmitted to an external electronic device (not shown in the Figs), enabling the external electronic device to read, analyze, and display the physiological information detected. Specifically, in the present embodiment, the carrying strap structure 1 includes two clasp assemblies 4 electrically connected to the two electrode structures 3 respectively. It is worth mentioning that, the clasp assembly 4 is formed of electrically conductive materials, and the clasp assembly 4 attached to the electrode structure 3 can provide electrical connection. The clasp assembly is, as a specific example, a rivet button.

Refer to FIG. 1E, which shows an exploded view of a combination of a signal transceiver and a clasp assembly. The sternum supporting harness S can further include a signal transceiver 5 electrically connected to the clasp assembly for use in conjunction with the clasp assembly 4. Specifically, the signal transceiver 5 can have a first signal transmitting terminal 51 and a second signal transmitting terminal 52 corresponding to the two clasp assemblies 4 respectively. The first signal transmitting terminal 51 and the second signal transmitting terminal 52 can respectively fit the apertures (not shown in the Figs) of the two clasp assemblies 4 to be fastened thereto. Thus, the first signal transmitting terminal 51 and the second signal transmitting terminal 52 can be electrically connected to the two clasp assemblies 4, the user's physiological information detected by the electrode structures 3 can consequently be transmitted to the signal transceiver 5. Therefore, the user's physiological information detected can be transmitted to the external electronic device, enabling the external electronic device to read, analyze, and display the physiological information detected. It is worth noting that, the carrying strap structure 1 can include a plurality of clasp assemblies 4 and a plurality of electrode structures 3 for detecting and transmitting the user's physiological information more accurately.

Referring to FIG. 1C and FIG. 1D, the carrying strap structure 1 further includes a loop unit 15 for detachably fixing the chest strap structure 2 on the carrying strap structure 1. The loop unit 15 includes a first loop portion 151 and a second loop portion 152. The first loop portion 151 is disposed on the left shoulder strap 121, and the second loop portion 152 is disposed on the right shoulder strap 122. The chest strap structure 2 can pass through the first loop portion 151 and the second loop portion 152 so as to be detachably attached to the carrying strap structure 1. In addition, the loop unit 15 may further include a third loop unit 153 for facilitating the chest strap structure 2 to be stably coupled to the carrying strap structure 1. The third loop 153 is disposed on the main body 11 and positioned at the rear of the main body 11, and the present disclosure is not limited thereto. In another embodiment, the chest strap structure 2 can be directly engaged on the carrying strap structure 1 through sewing and without the loop unit 15, or the chest strap structure 2 and the carrying strap structure 1 can be detachably coupled through magnets, buttons, or hook-and-loop fasteners and without the loop unit 15. In another exemplary embodiment, the loop unit 15 may include additional loop portions for detachably fixing the chest strap structure 2 on the carrying strap structure 1.

It is worth noting that, the first loop portion 151 can be formed with a first connecting element 1511, and the left shoulder strap 121 can be formed with a second connecting element 1512 for use in conjunction with the first connecting element 1511, enabling the user to attach the chest strap structure 2 on the carrying strap structure 1 or to detach the chest strap structure 2 from the carrying strap structure 1 more conveniently. The first connecting element 1511 on the first loop portion 151 corresponds to the second connecting element 1512, and the present disclosure is not limited thereto. The second loop portion 152 or the third loop portion 153 can have a similar configuration as the first loop portion 151. As a specific example, the first connecting element 1511 on the first loop portion 151 and the second connecting element 1512 can be a fastener having Velcro hooks and a fastener having Velcro loops, thus the first connecting element 1511 and the second connecting element 1512 can be detachably fastened to each other. For example, the first connecting element 1511 is a fastener having Velcro hooks, the second connecting element 1512 is a fastener having Velcro loops, and the present disclosure is not limited thereto.

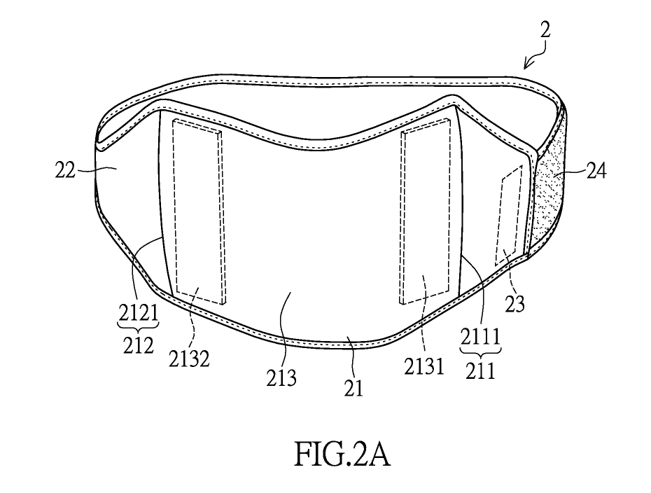

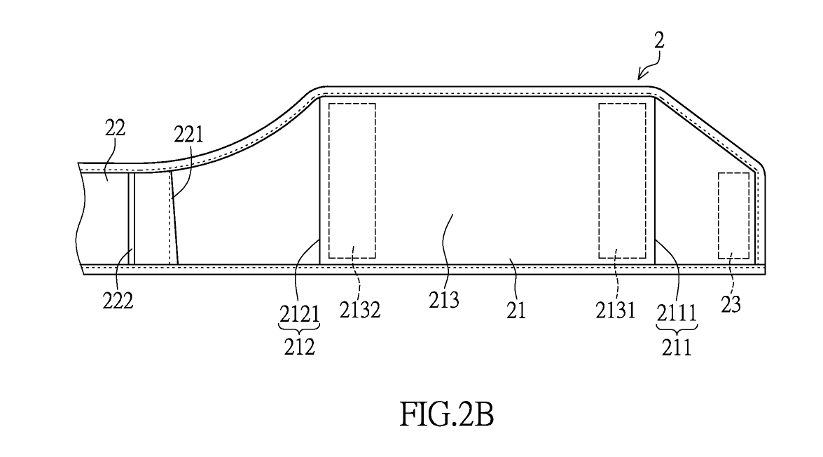

Refer to FIG. 2A and FIG. 2B. FIG. 2A shows a perspective view of a chest strap structure according to an embodiment of the present disclosure. FIG. 2B shows a front view of the chest strap structure according to an embodiment of the present disclosure. The chest strap structure 2 according to the present disclosure includes a grabbing unit 21 and a belt-like unit 22. The grabbing unit 21 is connected to the belt-like unit 22. For example, the grabbing unit 21 and the belt-like unit 22 are connected to each other though sewing. The chest strap unit 2 can be surroundingly disposed on the carrying strap structure 1 through the belt-like unit 22 and the grabbing unit 21 for supporting the user's sternum. To put it concretely, in the instant embodiment, the belt-like unit 22 of the chest strap structure 2 can sequentially pass through the second loop portion 152, the third loop 153, and the first loop portion 151, so as to be detachably attached to the carrying strap structure 1.

The chest strap structure 2 can be belt-like and have a first connecting element 23 and a second connecting element 24 for use in conjunction with the first connecting element 23. The chest strap structure 2 can be surroundingly disposed on the user's body through the first connecting element 23 and the second connecting element 24. As a specific example, the first connecting element 23 and the second connecting element 24 can be a fastener having Velcro hooks and a fastener having Velcro loops, thus the first connecting element 23 and the second connecting element 24 can be detachably fastened to each other. For example, the first connecting element 23 is a fastener having Velcro hooks, the second connecting element 24 is a fastener having Velcro loops, and the present disclosure is not limited thereto.

The grabbing unit 21 includes a first grabbing portion 211, a second grabbing portion 212, and a base portion 213. The first grabbing portion 211 and the second gabbing portion 212 are correspondingly arranged with respect to each other, and the first grabbing portion 211 and the second grabbing portion 212 are respectively positioned at two opposite sides of the base portion 213. The grabbing unit 21 is formed with a first opening 2111 between the first grabbing portion 211 and the base portion 213, and the grabbing unit 21 is formed with a second opening 2121 between the second grabbing portion 212 and the base portion 213. The first opening 2111 and the second opening 2121 respectively face toward two opposite directions, such that the first opening 2111 and the second opening 2121 can form a pocket-like structure on the base portion 213. The distance between the first grabbing portion 211 and the second grabbing portion 212 can be about the size of the user's palm, enabling the user, who is wearing the sternum supporting harness S, to grab the first grabbing portion 211 and the second grabbing portion 212 with a single hand to utilize the chest strap structure 2 to support the sternum. To put it concretely, when using one hand to grab the grabbing unit 21 for supporting the sternum, the user can insert his thumb into the first opening 2111 to grab the first grabbing portion 2111, and the user can insert his other fingers into the second opening 2121 to grab the second grabbing portion 212. It is worth noting that, because the sizes of the users' palms can be different, the distance between the first grabbing portion 211 and the second grabbing portion 212 can be sized according to the needs.

The grabbing unit 21 may further include a first reinforcement piece 2131 and a second reinforcement piece 2132 for reinforcing the support of the chest strap structure 2 when grabbed. The first reinforcement piece 2131 and the second reinforcement piece 2132 are disposed inside the base portion 213. The first reinforcement piece 2131 and the first grabbing portion 211 are correspondingly arranged with respect to each other. The second reinforcement piece 2132 and the second grabbing portion 212 are correspondingly arranged with respect to each other. Specifically, in the present embodiment, the first reinforcement piece 2131 is positioned under the first grabbing portion 211 and proximal to the user's left flank. The second reinforcement piece 2132 is positioned under the second grabbing portion 212 and proximal to the user's right flank. The first reinforcement piece 2131 and the second reinforcement 2132 can each be formed of a stiff plate, such as an acrylic plate or a sheet having small resilience, for reinforcing the support of the chest strap structure 2 when the grabbing unit 21 is grabbed. The lateral width of the first reinforcement piece 2131 can be approximately equal to the depth of the first opening 2111, and the lateral width of the second reinforcement piece 2132 can be approximately equal to the depth of the second opening 2121. It is worth noting that, the chest strap structure 2 can be formed of materials having predetermined strength and small resilience for supporting the user's sternum to prevent the wound on the user's body from being torn and to prevent the sternum from fracturing. Moreover, when the user grabs the grabbing unit 21, the supporting force provided by the chest strap structure 2 will not be affected by the grabbing.

The chest strap structure 2 may further include a folding portion 221 disposed on the belt-like unit 22 for preventing the belt-like unit 22 passing through the first loop portion 151, the second loop portion 152, and the third loop portion 153 from curling, and for preventing the front-side of the belt-like unit 22 from being turned backward or vice versa. The folding portion 221 can be formed by bending the belt-like unit 22 at a predetermined angle, such that portions of the belt-like unit 22 overlap, and then stitching the overlapped portions of the belt-like unit 22. The folding portion 221 is thicker than the initial belt-like material that forms the belt-like unit 22. The folding portion 221 facilitates the belt-like unit 22 to be maintained unfolded. In addition, the belt-like unit 22 may further include a reinforcement piece 222 disposed inside the folding portion 221 and fixed therein by stitching. The reinforcement piece 222 can be formed of a stiff sheet for increasing the stiffness the belt-like unit 22.

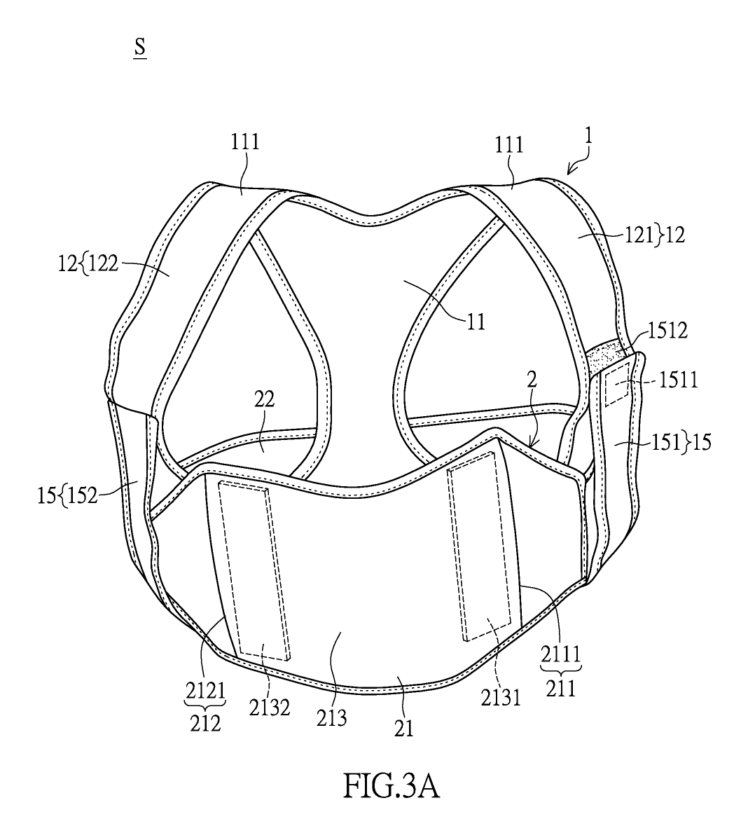

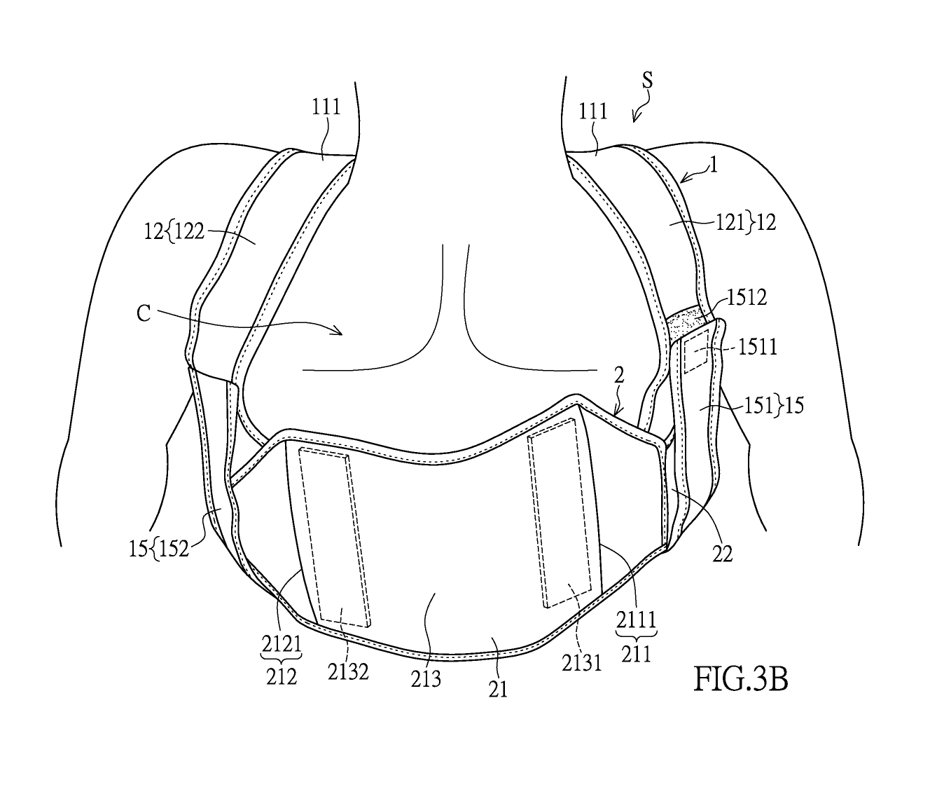

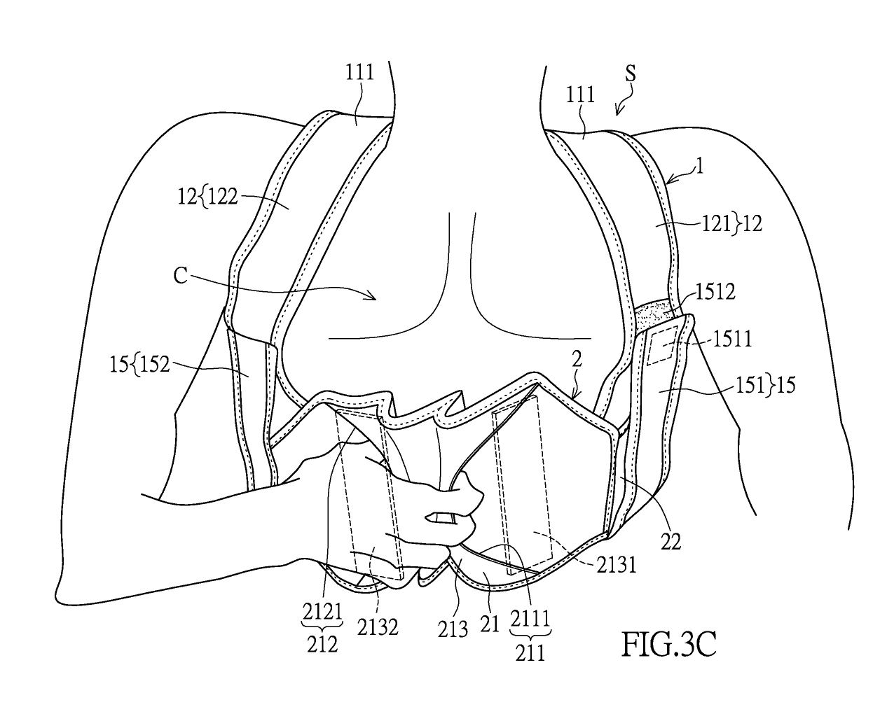

Refer to FIG. 3A to FIG. 3C. FIG. 3A shows a perspective view of a sternum supporting harness according to an embodiment of the present disclosure. FIG. 3B shows a perspective view of the sternum supporting harness in use according to an embodiment of the present disclosure. FIG. 3C shows another perspective view of the sternum supporting harness in use according to an embodiment of the present disclosure. The belt-like unit 22 of the chest strap structure 2 can pass through the second loop portion 152, the third loop portion 153, and the first loop portion 151 to be detachably attached to the carrying strap structure 1, forming the sternum supporting harness S of the present disclosure. As shown in FIG. 3B and FIG. 3C, the user wearing the sternum supporting harness S can grab the first grabbing portion 211 and the second grabbing portion 212 for supporting the sternum C, thus to prevent the wound from being torn.

To sum up, the sternum supporting harness S according to the present disclosure including the carrying strap structure 1 and the chest strap structure 2 detachably attached thereto can be two-piece worn on the user. The sternum supporting harness S can support the user's sternum to prevent the wound on the chest from being torn when the user grabs the first grabbing portion 211 and the second grabbing portion 212. The electrode structures 3 and the clasp assemblies 4 disposed on the carrying strap structure 1 is for use in conjunction with the signal transceiver 5, for recording the user's physiological information and transmitting the information to the external electronic device. Moreover, the sternum supporting harness S utilizes the first grabbing portion 211 and the second grabbing portion 212 to simplify the design, and the sternum supporting harness S is configured to be two-piece worn on the user through the carrying strap structure 1 and the chest strap structure 2, where the first grabbing portion 211 and the second grabbing unit 212 of the grabbing unit 21 each are a non-grabbing-ring design. Therefore, the sternum supporting harness S can be worn inside the user's clothing and in direct contact with the user's skin, for providing preferred support to the user's sternum C, and the user does not receive any strange looks.

The descriptions illustrated supra set forth simply the preferred embodiments of the present disclosure; however, the characteristics of the present disclosure are by no means restricted thereto. All changes, alterations, or modifications conveniently considered by those skilled in the art are deemed to be encompassed within the scope of the present disclosure delineated by the following claims.

What is claimed is:

1. A sternum supporting harness for supporting a sternum, comprising:
a carrying strap structure including a main body, a shoulder portion, a flank portion, and an adjustment portion, wherein the main body has two upper end portions respectively positioned at opposite sides of the main body, the shoulder portion includes a left shoulder strap and a right shoulder strap, an end of the left shoulder strap and an end of the right shoulder strap are connected to the upper end portions respectively, and another end of the left shoulder strap and another end of the right shoulder strap are connected to the flank portion, wherein the adjustment portion is connected to the flank portion for facilitating the main body to be disposed on the sternum; and
a chest strap structure including a grabbing unit and a belt-like unit, wherein the grabbing unit is connected to the belt-like unit, and the chest strap structure is surroundingly disposed on the carrying strap structure through the belt-like unit and the grabbing unit, wherein the grabbing unit includes a first grabbing portion, a second grabbing unit, and a base portion, the first grabbing portion and the second grabbing portion are correspondingly arranged with respect to each other, and the first grabbing portion and the second grabbing portion are respectively positioned at two opposite sides of the base portion;
wherein the sternum supporting harness is for supporting the sternum when the first grabbing portion and the second grabbing portion are grabbed.

2. The sternum supporting harness of claim 1, wherein the grabbing unit is formed with a first opening between the first grabbing portion and the base portion and with a second opening between the second grabbing portion and the base portion, and the first opening and the second opening respectively face toward two opposite directions.

3. The sternum supporting harness of claim 1, wherein the carrying strap structure further includes a loop unit including a first loop portion and a second loop portion, wherein the first loop portion is disposed on the left shoulder strap, the second loop portion is disposed on the right shoulder strap, and the chest strap structure passes through the first loop portion and the second loop portion so as to be detachably attached to the carrying strap structure.

4. The sternum supporting harness of claim 1, wherein the flank portion includes a left flank strap and a right flank strap, another end of the left shoulder strap is connected to the left flank strap, and another end of the right shoulder strap is connected to the right flank strap.

5. The sternum supporting harness of claim 1, wherein the adjustment portion is formed with a first connecting element and a second connecting element, and the adjustment portion facilitates the carrying strap structure to be disposed on the sternum through the first connecting portion and the second connecting portion.

6. The sternum supporting harness of claim 1, wherein the chest strap structure further includes a folding portion disposed on the belt-like unit.

7. The sternum supporting harness of claim 1, wherein the grabbing unit further includes a first reinforcement piece and a second reinforcement piece disposed inside the base portion, the first reinforcement piece and the first grabbing portion are correspondingly arranged with respect to each other, and the second reinforcement piece and the second grabbing portion are correspondingly arranged with respect to each other.

8. The sternum supporting harness of claim 1, further comprising an electrode structure disposed on the flank portion.

9. The sternum supporting harness of claim 8, further comprising a clasp assembly disposed on the main body, wherein the electrode structure is electrically connected to the clasp assembly.

10. The sternum supporting harness of claim 9, further comprising a signal transceiver electrically connected to the clasp assembly.
